(19) United States
(12) Patent Application Publication
(10) Pub. No.: US 2016/0089437 A1
(43) Pub. Date: Mar. 31, 2016

(54) FLOCCULATED MEGESTROL ACETATE SUSPENSION

(71) Applicant: Sou Yung Hsiao, East Northport, NY (US)

(72) Inventor: Yih Ming HSIAO, East Northport, NY (US)

(21) Appl. No.: 14/499,744

(22) Filed: Sep. 29, 2014

(51) Int. Cl. A61K 47/10; A61K 31/57

(52) U.S. Cl. CPC A61K 47/10; A61K 31/57

(57) ABSTRACT

The present invention relates to a novel oral pharmaceutical composition in the form of a stable flocculated suspension in water, said composition comprising micronized megestrol acetate at a concentration of 10 to 200 mg per ml, polysorbate at a concentration of 0.01% to 12.6% weight by volume, or polyethylene glycol at a concentration of about 0.01% to 5.0% weight by volume, polyoxyl 35 castor oil at a concentration of 0.05% to 51.5% weight by volume and/or polyoxyl 40 hydrogenated castor oil at a concentration of 0.05% to 45% weight by volume in a stable flocculated suspension in water. The composition also may include preservatives, sweeteners, pH adjusting agents and flavoring agents.

### FIELD OF THE INVENTION

The present invention relates to a pharmaceutical composition comprising micronized megestrol acetate which is the generic name for 17-alpha-acetyloxy-6-methylpregna-4,6-diene-3,20-dione.

### BACKGROUND OF THE INVENTION

Megestrol acetate is a synthetic derivative of the naturally occurring steroid hormone, progesterone. U.S. Pat. No. 5,338,732 to Atzinger et al. discloses an oral pharmaceutical composition of micronized megestrol acetate at a concentration of 15 to 150 mg/ml, preferably 20 to 120 mg/ml, and most preferably 40 mg/ml in combination with a polysorbate at a concentration of 0.005% to 0.015% weight/volume (w/v) and a polyethylene glycol at a concentration greater than 5% (w/v), wherein said composition forms a stable flocculated suspension in water. In the most preferred formulation of U.S. Pat. No. 5,338,732, the internal phase comprises micronized megestrol acetate having a mass median diameter between 3 and 10 microns; and the external phase is composed essentially of purified water plus polysorbate 80, polyethylene glycol 1450, xanthan gum, sodium benzoate, citric acid, sodium citrate, sucrose and flavors. The product with a proprietary name Megace is an anti-neoplastic drug.

U.S. Pat. No. 3,356,573 to Kirk et al. discloses a megestrol acetate pharmaceutical tablet preparation comprising lactose, magnesium stearate and starch. Kirk et al. also discloses that liquid compositions of megestrol acetate can take the form of solutions, emulsions, suspension, syrups and elixirs but does not provide details of the compositions of such formulations.

U.S. Pat. No. 4,396,615 to Petrow et al. discloses a method for treating androgen-related disorders by administering to a patient 6-methylene progesterone derivatives together with a megestrol acetate formulation, but does not elaborate on what constitutes the megestrol acetate formulation comprises.

U.S. Pat. No. 4,370,321 to Greaney et al. discloses an adjuvant therapy for the treatment of breast cancer by employing megestrol acetate. However, the type or composition of the megestrol acetate formulation is not specifically described.

U.S. Pat. No. 4,666,885 to Labrie et al. discloses a combination therapy for the treatment of female breast cancer comprising administrating to a patient luteinizing hormones in combination with anti-androgen agents such as megestrol acetate. In particular, Labrie et al. disclose that the anti-androgen agents are formulated with conventional pharmaceutical excipients (e.g., spray dried lactose and magnesium stearate) into tablets or capsules for oral administration.

U.S. Pat. No. 4,760,053 to Labrie et al. discloses a treatment for sex steroid dependent cancers by a combination therapy which includes the use of megestrol acetate, but does not describe the type or composition of the pharmaceutical formulation used in the treatment.

U.S. Pat. No. 4,775,661 to Labrie et al. discloses a combination therapy for the treatment of female breast cancer in which megestrol acetate is disclosed as a suitable steroidal anti-androgen agent. It is disclosed that megestrol acetate, as an active substance, may be mixed with binders, such as polyethylene glycol, and taste improving substances, to form tablets or dragee cores.

J. H. Von Roenn et al. described in Annals of Internal Medicine, 109, 840-841 (1988) use of megestrol acetate for the treatment of cachexia associated with human immunodeficiency virus (HIV) infection. The dose of megestrol acetate was reported but the type or composition of the formulation was not mentioned.

U.S. Pat. No. 6,028,065 to Ragunathan et al. discloses an oral pharmaceutical composition comprising megestrol acetate at a concentration of 2 to 6%, preferably about 4%, but having a composition different from that described in U.S. Pat. No. 5,338,732 to Atzinger et al. Specifically, Raguathan et al. disclose a stable flocculated suspension in water comprising megestrol acetate, at least one compound selected from polyethylene glycol, propylene glycol, glycerol and sorbitol and a surfactant, wherein polysorbate and polyethylene glycol are not simultaneously present in the composition. Ragunathan et al. disclose that the compound selected from polyethylene glycol, propylene glycol, glycerol and sorbitol is preferably used at a concentration of up to 40% w/v, more preferably 5 to 30% w/v, and most preferably 10 to 25% w/v, and that the surfactant is preferably used at a concentration of about 0.0001 to 0.03% w/v. Ragunathan et al. also disclose the use of a suspending agent (xanthan gum), preferably at a concentration of about 0.1 to 0.35% w/v, more preferably about 0.15 to 0.25%.

U.S. Pat. No. 6,268,356 to Ragunathan et al. discloses an oral stable flocculated suspension comprising micronized megestrol acetate at a concentration of 10 mg to 200 mg per ml, wherein a compound selected from polyethylene glycol, propylene glycol, glycerol and sorbitol is used at a concentration of 10 to 40% by weight, and a surfactant is used at a concentration of 0.0001 to 0.03% by weight.

Ragunathan et al, U.S. Pat. No. 6,593,318, discloses a stable flocculated suspension of about 10 to 200 mg per ml of micronized megestrol acetate, about 10 to 40% by weight of at least one compound selected from the group consisting of polyethylene glycol, propylene glycol, glycerol and sorbitol, and about 0.0001 to 0.03% by weight of a surfactant, wherein polysorbate and polyethylene glycol are not simultaneously present in said composition.

U.S. Pat. No. 6,593,320 to Ragunathan et al. discloses an oral pharmaceutical composition in the form of a stable flocculated suspension in water and being prepared by a method comprising: forming a solution by combining water with a surfactant and at least one compound selected from the group consisting of polyethylene glycol, propylene glycol, glycerol and sorbitol, wherein the total concentration of all compounds is up to 40% weight/volume, provided that polyethylene glycol and polysorbate are not simultaneously present in the composition; and combining the solution with about 10 to 200 mg per ml of megestrol acetate.

U.S. Pat. No. 7,101,576 to Hovey et al. discloses nanoparticulate compositions comprising megestrol. The megestrol particles of the composition have an effective average particle size of less than about 2000 nm.

Par pharmaceutical, Inc., Spring Valley, N.Y. on Sep. 5, 2006 announced that a new patent (U.S. Pat. No. 7,101,576) has been issued by the U.S. Patent and Trademark Office relating to Megace® ES (megestrol acetate) 625 mg/5 ml oral suspension, which utilizes Elan's NanoCrysta, Technology delivery system.

There is a continuing need in the art for pharmaceutical elegant and stable oral suspension dosage forms of megestrol acetate, especially for higher doses.

### SUMMARY OF THE INVENTION

The object of the present invention is to provide an oral pharmaceutical elegant and stable flocculated suspension of micronized megestrol acetate which is different from the known formulations described in the background of the invention above and which provides similar in-vitro dissolution performance to Megace ES oral suspension from Par Pharmaceutical, Inc.

In one embodiment, the present invention provides an oral pharmaceutical elegant and stable composition, which may be in the form of a flocculated suspension of micronized megestrol acetate and comprises:

(a) about 10 to 200 mg per ml of micronized megestrol acetate;

(b) about 0.01 to 55% weight/volume of at least one wetting agent; and

(c) about 0.2 to 1.5% weight/volume of one or two suspending agents,

wherein the pH of the suspension can be controlled to 3.0˜4.7 by using an appropriate organic acid.

The oral pharmaceutical composition of the present invention is useful in the treatment of anorexia, cachexia or significant weight loss resulting from or associated with acquired immunodeficiency syndrome (AIDS).

### BRIEF DESCRIPTION OF THE FIGURES

FIG. 1 shows that the dissolution of the megestrol acetate suspension of Example 1 is substantially equivalent to that of Par's Megace® ES megestrol acetate suspension.

FIG. 2 shows that the dissolution of megestrol acetate suspension of Example 4 is substantially equivalent to that of Par's Megace® ES megestrol acetate suspension.

FIG. 3 shows that the dissolutions of megestrol acetate suspensions (FT-024/with homogenization, and FT-024/without homogenization) of Example 6 are substantially equivalent to that of Par's Megace® ES megestrol acetate suspension.

FIG. 4 shows that the dissolutions of megestrol acetate suspension of Example 9 is substantially equivalent to that of Par's Megace® ES megestrol acetate suspension.

### DETAILED DESCRIPTION OF THE INVENTION

In an embodiment of the invention, there is provided an oral pharmaceutical elegant composition comprising micronized megestrol acetate at a concentration of 10 to 200 mg per ml, preferably about 10 to 150 mg per ml, and most preferably about 125 mg per ml. It is preferable that the megestrol acetate is micronized so that about 90% by weight of particles is below 20 microns and the mass median diameter is between 3 to 10 microns and that the micronized particles may be dispersed in water with wetting agents, such as a combination of polysorbate 80, polyoxyl 35 castor oil, and polyoxyl 40 hydrogenated castor oil.

In one embodiment of the present invention, the wetting agents in the composition have at least one straight carbon chain of up to 40, preferably 4-40, atoms in the hydrophobic group.

The wetting agents in the composition of the present invention may include polyoxylene sorbitan fatty acid esters, such as polysorbate 80, polyoxyethylene castor oil derivatives, such as polyoxyl 35 castor oil, and polyoxyl 40 hydrogenated castor oil, poloxamer, polyoxyethylene alkyl ethers, polyethylene glycol and polyoxyethylene stearates. The concentration of the wetting agents in the composition may be about 0.01 to 55% weight/volume, preferably about 0.05% to 51.5% weight by volume. For example, the wetting agent may be polyoxyl 35 castor oil at a concentration of about 0.05% to 51.5% weight by volume; or may be polyoxyl 40 hydrogenated castor oil at a concentration of about 0.05% to 45% weight by volume; or may be polysorbate 80 at a concentration of about 0.01% to 12.6% weight by volume; or may be polyethylene glycol 400 at a concentration of about 0.01% to 5.0%, weight by volume.

A hydrophobic solid of megestrol acetate is not easily wetted by water. The hydrophobic solid particles of megestrol acetate in the suspension are only hydrated slightly or not at all because water molecules prefer to interact with one another instead of solvating the particles. Hence, such particles do not disperse or dissolve spontaneously in water. Surfactants or wetting agents may be needed to provide a suspension and maintain the physical stability.

The suspending agents in the composition of the present invention may include colloidal silicon dioxide, carboxymethylcellulose sodium, dextrin, gelatin, microcrystalline cellulose, povidone, xanthan gum, tragacanth and a mixture of microcrystalline cellulose and carboxymethyl cellulose sodium.

The suspending agent is used to maintain the megestrol acetate particles in a uniform suspension stayed for a longer period of time and during the dose administration period. In order to achieve this purpose, the suspending agents are present in the composition at a concentration of about 0.02 to 4.0% weight/volume, preferably at a concentration of about 0.05 to 2.0% weight/volume, most preferably at a concentration of about 0.2 to 1.5% weight/volume. The suspending agent may be at a concentration of about 0.2 to 1.5% weight/volume and is a mixture of microcrystalline cellulose and carboxymethyl cellulose; or is at a concentration of 0.1 to 2.0% weight/volume and is colloidal silicone dioxide.

The pH of the suspension should be adequately adjusted in order to make the suspension stable and effective. In one embodiment of the present invention, the pH of the megestrol acetate suspension is preferably about 3.0 to 4.7. The pH of the suspension may be adjusted by using one or more organic acids, such as citric acid.

The composition of the present invention may comprise conventional additives including preservatives, pH adjusting agents, sweeteners, flavoring agents and ant-foaming agents, etc., which may be present at the following concentrations:
preservative: sodium benzoate at 0.1˜0.5% w/v,
pH adjusting agent: citric acid at about 0.3˜0.6% w/v
sweeteners: sodium saccharine at 0.4˜1.0% w/v, glycerin at 6˜10% w/v and sorbitol at 1˜30% w/v,
flavoring agent: banana flavor at about 0.1˜2% w/v
anti-foaming agents: simethicone (or dimethicone mixed with silicon dioxide) at about 0.2˜2% w/v.

In one preferred embodiments, the oral pharmaceutical composition comprises, on a percent weight/volume basis, megestrol acetate 12.5%, microcrystalline cellulose and carboxymethylcellulose sodium 0.8%, glycerin 8%, sodium benzoate 0.2%, polyoxyl 35 castor oil 15%, polyoxyl 40 hydrogenated castor oil 10%, sodium saccharine 0.4%, citric acid 0.32%, polysorbate 80 5%, 70% sorbitol solution 20.0%, banana flavor 0.5%, a mixture of dimethicone 0.95% and colloidal silicon dioxide 0.05%, and the remainder purified water, wherein the composition forms a stable flocculated suspension in water.

In one preferred embodiment, the oral pharmaceutical composition comprises, on a percent weight/volume basis, megestrol acetate 12.5%, dimethicone 0.7%, colloidal silicon dioxide 0.5%, glycerin 8.0%, sodium benzoate 0.2%, polyoxyl 35 castor oil 15.0%, polyoxyl 40 hydrogenated castor oil 10.0%, sodium saccharine 0.5%, citric acid 0.5%, polysorbate 80 5%, 70% sorbitol solution 2%, banana flavor 1.0%, and the remainder purified water, wherein the composition forms a stable flocculated suspension in water.

In another preferred embodiment, the oral pharmaceutical composition comprising on a percent weight/volume basis megestrol acetate 4.0%, dimethicone 0.5%, colloidal silicon dioxide 2.0%, polyoxyl 35 castor oil 30.0%, sodium benzoate 0.2%, sodium saccharine 0.5%, citric acid 0.5%, polyethylene glycol 5%, 70% sorbitol solution 2%, banana flavor 1.0%, and the remainder purified water, wherein the composition forms a stable flocculated suspension in water.

In still another preferred embodiment, the oral pharmaceutical composition comprises, on a percent weight/volume basis, megestrol acetate 12.5%, dimethicone 0.5%, colloidal silicon dioxide 2.0%, glycerin 8.0%, sodium benzoate 0.2%, polyoxyl 35 castor oil 33.0%, sodium saccharine 0.5%, citric acid 0.5%, polyethylene glycol 5%, 70% sorbitol solution 2%, banana flavor 1.0%, and the remainder purified water, wherein the composition forms a stable flocculated suspension in water.

In a further preferred embodiment, the oral pharmaceutical composition comprising on a percent weight/volume basis megestrol acetate 4.0%, dimethicone 0.5%, colloidal silicon dioxide 1.0%, glycerin 8%, sodium benzoate 0.2%, polyoxyl 35 castor oil 12.0%, citric acid 0.5%, sodium saccharine 0.45%, polyethylene glycol 5%, 70% sorbitol solution 2%, banana flavor 1.0%, and the remainder purified water, wherein the composition forms a stable flocculated suspension in water.

### Example 1

### Megestrol Acetate Composition

A banana flavored oral suspension containing 125 mg of megestrol acetate per milliliter has the following composition.

| | Quantity | | |
|---|---|---|---|
| Ingredient | per ml | | % w/v |
| Megestrol acetate, micronized | 125 | mg | 12.5 |
| microcrystalline cellulose and | 8 | mg | 0.8 |
| carboxymethylcellulose sodium | | | |
| Glycerin | 80 | mg | 8.0 |
| Sodium benzoate | 2 | mg | 0.2 |
| Polyoxyl 35 castor oil | 150 | mg | 15.0 |
| Citric acid | 3.2 | mg | 0.32 |
| Sodium saccharin | 4 | mg | 0.4 |
| Polyoxyl 40 hydrogenated castor oil | 100 | mg | 10.0 |
| Sorbitol solution, 70% | 200 | mg | 20.0 |
| Polysorbate 80 | 50 | mg | 5.0 |
| Banana flavor | 5 | mg | 0.5 |
| Purified water q.s. | 1 | ml | 100 |

Preparation of the megestrol acetate suspension, using proportional amounts of the ingredients, is carried out as follows:

Approximately one quarter of purified water is mixed with microcrystalline cellulose and carboxymethylcellulose sodium, sorbitol solution and glycerin to form a suspension. Polyoxyl 35 castor oil, polyoxyl 40 hydrogenated castor oil, polysorbate 80 and megestrol acetate are mixed till homogeneous to form another suspension. The two suspensions are mixed together well. Citric acid, sodium saccharine, sodium benzoate and banana flavor are added into the suspension and mixed well. Purified water is added and adjusted to the desired volume. The entire suspension is then passed through a colloid mill or homogenizer to provide an oral suspension containing 40 mg/ml of megestrol acetate. Suspensions containing from 20 to 200 mg/ml of megestrol acetate are prepared in the similar manner.

### Example 2

### Comparative Dissolutions of Suspensions

One of the major considerations in the comparative dissolutions of the megestrol acetate suspension with the Megace® ES megestrol acetate suspension is the tendency of the suspensions' bioequivalency. A method for evaluation is measurement of the percent of megestrol acetate absorbed in the dissolution medium at the various periods of time.

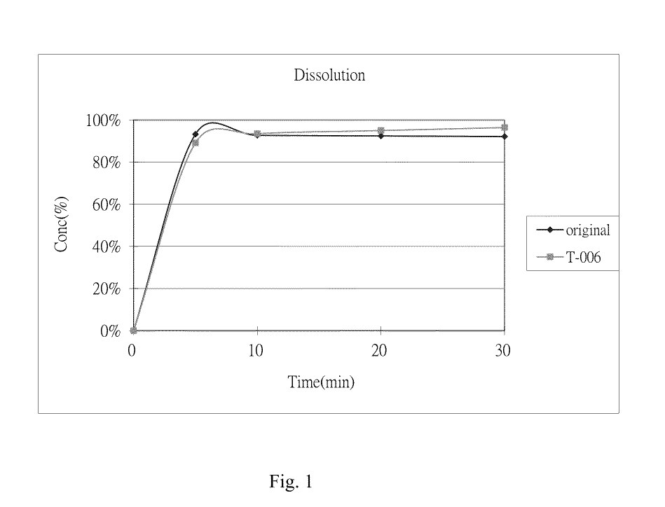

The comparative dissolutions of megestrol acetate suspension, 625 mg/5 ml and Megace® ES megestrol acetate suspension in the 0.5% sodium lauryl sulfate solution at 37° C. and under 25 rpm rotation have the profiles shown in FIG. 1. FIG. 1 shows that the dissolution of the megestrol acetate suspension is substantially equivalent to that of Par's Megace® ES megestrol acetate suspension.

In FIG. 1, “original” is Megace® ES megestrol acetate oral suspension from Par Pharmaceutical, Inc., Spring Valley, N.Y., Control No: 22697201 and Exp. Date: July 2013. “T-006” is megestrol acetate oral suspension prepared from Example 1.

### Example 3

A banana flavored oral suspension containing 40 mg of megestrol acetate per milliliter has the following composition.

| | Quantity | | |
|---|---|---|---|
| Ingredient | per ml | | % w/v |
| Megestrol acetate, micronized | 40 | mg | 4.0 |
| microcrystalline cellulose and | 8 | mg | 0.8 |
| carboxymethylcellulose sodium | | | |
| Glycerin | 80 | mg | 8.0 |
| Sodium benzoate | 2 | mg | 0.2 |
| Polyoxyl 35 castor oil | 150 | mg | 15.0 |
| Citric acid | 3.2 | mg | 0.32 |
| Sodium saccharin | 4 | mg | 0.4 |
| Polyoxyl 40 hydrogenated castor | 100 | mg | 10.0 |
| oil | | | |
| Sorbitol solution, 70% | 200 | mg | 20.0 |
| Polysorbate 80 | 50 | mg | 5.0 |
| Banana flavor | 5 | mg | 0.5 |
| Purified water q.s. | 1 | ml | 100 |

### Example 4

### Megestrol Acetate Composition

A banana flavored oral suspension containing 125 mg of megestrol acetate per milliliter has the following composition.

| | Quantity | | |
|---|---|---|---|
| Ingredient | per ml | | % w/v |
| Megestrol acetate, micronized | 125 | mg | 12.5 |
| microcrystalline cellulose and | 8 | mg | 0.8 |
| carboxymethylcellulose sodium | | | |
| Glycerin | 80 | mg | 8.0 |
| Sodium benzoate | 2 | mg | 0.2 |
| Polyoxyl 35 castor oil | 150 | mg | 15.0 |
| Citric acid | 3.2 | mg | 0.32 |
| Sodium saccharin | 4 | mg | 0.4 |
| Polyoxyl 40 hydrogenated castor oil | 100 | mg | 10.0 |
| Sorbitol solution, 70% | 200 | mg | 20.0 |
| Polysorbate 80 | 50 | mg | 5.0 |
| Banana flavor | 5 | mg | 0.5 |
| Dimethicone | 9.5 | mg | 0.95 |
| Colloidal silicone dioxide | 0.5 | mg | 0.05 |
| Purified water q.s. | 1 | ml | 100 |

### Example 5

### Comparative Dissolutions of Suspensions

One of the major considerations in the comparative dissolutions of the megestrol acetate suspension with the Megace® ES megestrol acetate suspension is the tendency of the suspensions' bioequivalency. A method for evaluation is measurement of the percent of megestrol acetate absorbed in the dissolution medium at the various periods of time.

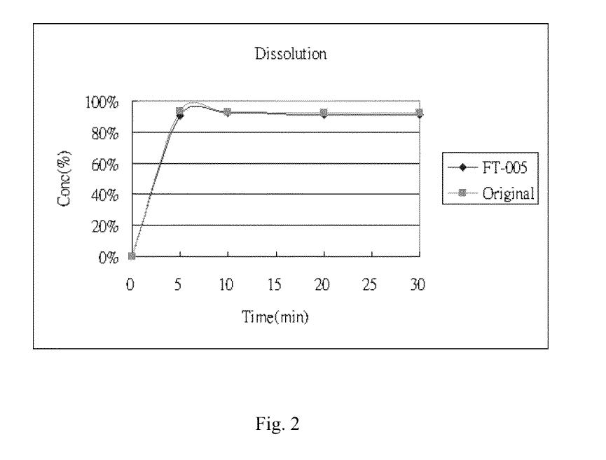

The comparative dissolutions of megestrol acetate suspension, 625 mg/5 ml and Megace® ES megestrol acetate suspension in the 0.5% sodium lauryl sulfate solution at 37° C. and under 25 rpm rotation have the profiles shown in FIG. 2. FIG. 2 shows that the dissolution of megestrol acetate suspension is substantially equivalent to that of Par's Megace® ES megestrol acetate suspension.

In FIG. 2, “Original” is Megace® ES megestrol acetate oral suspension from Par Pharmaceutical, Inc., Spring Valley, N.Y., Control No: 22697201 and Exp. Date: July 2013. “FT-005” is megestrol acetate oral suspension prepared from Example 4.

### Example 6

### Megestrol Acetate Composition

A banana flavored oral suspension containing 125 mg of megestrol acetate per milliliter has the following composition.

| | Quantity | | |
|---|---|---|---|
| Ingredient | per ml | | % w/v |
| Megestrol acetate, micronized | 125 | mg | 12.5 |
| Glycerin | 80 | mg | 8.0 |
| Sodium benzoate | 2 | mg | 0.2 |
| Polyoxyl 35 castor oil | 150 | mg | 15.0 |
| Citric acid | 5.0 | mg | 0.50 |
| Sodium saccharin | 5 | mg | 0.5 |
| Polyoxyl 40 hydrogenated castor oil | 100 | mg | 10.0 |
| Sorbitol solution, 70% | 20 | mg | 2.0 |
| Polysorbate 80 | 50 | mg | 5.0 |
| Banana flavor | 10 | mg | 1.0 |
| Dimethicone | 7.0 | mg | 0.7 |
| Colloidal silicone dioxide | 5.0 | mg | 0.5 |
| Purified water q.s. | 1 | ml | 100 |

### Example 7

### Comparative Dissolutions of Suspensions

One of the major considerations in the comparative dissolutions of the megestrol acetate suspension prepared from Example 6 (FT-024/with homogenization, and FT-024/without homogenization) with Par's Megace® ES megestrol acetate suspension (Control No: 22697201 and Exp. Date: July 2013) is the tendency of the suspensions' bioequivalency.

A method for evaluation is measurement of the percent of megestrol acetate absorbed in the dissolution medium at the various periods of time.

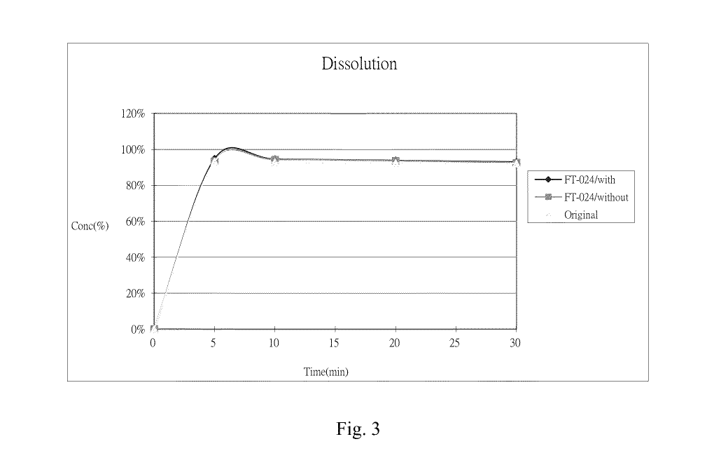

The comparative dissolutions of megestrol acetate suspension, 625 mg/5 ml (FT-024/with homogenization, and FT-024/without homogenization) and Par's Megace® ES megestrol acetate suspension (Control No: 22697201 and Exp. Date: July 2013) in the 0.5% sodium lauryl sulfate solution at 37° C. and under 25 rpm rotation have the profiles shown in FIG. 3. FIG. 3 shows that the dissolutions of megestrol acetate suspensions (FT-024/with homogenization, and FT-024/without homogenization) are substantially equivalent to that of Par's Megace® ES megestrol acetate suspension.

In FIG. 3, “FT-024/with” means FT-024/with homogenization, “FT-024/without” means FT-024/without homogenization, and “Original” means Par's Megace® ES megestrol acetate suspension.

### Example 8

### Megestrol Acetate Composition

A banana flavored oral suspension containing 40 mg of megestrol acetate per milliliter has the following composition.

| | Quantity | | |
|---|---|---|---|
| Ingredient | per ml | | % w/v |
| Megestrol acetate, micronized | 40 | mg | 4.0 |
| Glycerin | 80 | mg | 8.0 |
| Sodium benzoate | 2 | mg | 0.2 |
| Polyoxyl 35 castor oil | 120 | mg | 12.0 |
| Citric acid | 5.0 | mg | 0.50 |
| Sodium saccharin | 5 | mg | 0.45 |
| Sorbitol solution, 70% | 20 | mg | 2.0 |
| Polyethylene glycol 400 | 50 | mg | 5.0 |
| Banana flavor | 10 | mg | 1.0 |
| Dimethicone | 5.0 | mg | 0.5 |
| Colloidal silicone dioxide | 10.0 | mg | 1.0 |
| Purified water q.s. | 1 | ml | 100 |

### Example 9

### Megestrol Acetate Composition

A banana flavored oral suspension containing 125 mg of megestrol acetate per milliliter has the following composition.

| | Quantity | | |
|---|---|---|---|
| Ingredient | per ml | | % w/v |
| Megestrol acetate, micronized | 125 | mg | 12.5 |
| Glycerin | 80 | mg | 8.0 |
| Sodium benzoate | 2 | mg | 0.2 |
| Polyoxyl 35 castor oil | 300 | mg | 30.0 |
| Citric acid | 5.0 | mg | 0.50 |
| Sodium saccharin | 5 | mg | 0.5 |
| Sorbitol solution, 70% | 20 | mg | 2.0 |
| Polyethylene glycol 400 | 50 | mg | 5.0 |
| Banana flavor | 10 | mg | 1.0 |
| Dimethicone | 5.0 | mg | 0.5 |
| Colloidal silicone dioxide | 20.0 | mg | 2.0 |
| Purified water q.s. | 1 | ml | 100 |

### Example 10

### Dissolution of Suspension

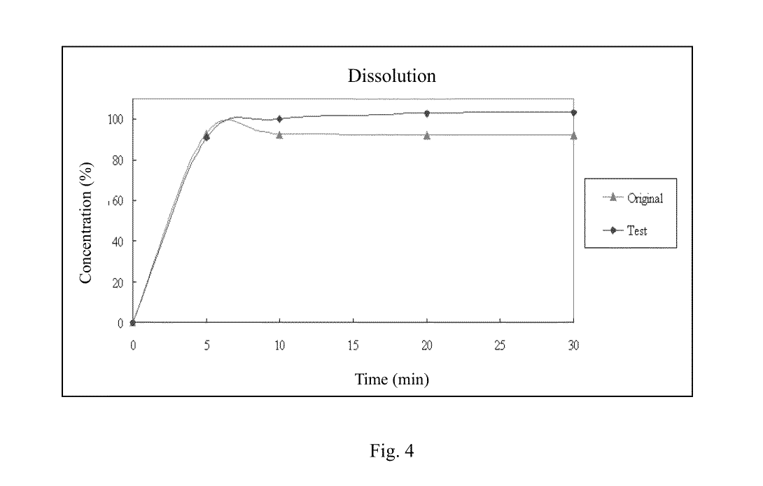

The comparative dissolutions of megestrol acetate oral suspension, 125 mg/ml from Example 9, and of Par's Megace®ES megestrol acetate suspension, in the 0.5% sodium lauryl sulfate solution at 37° C. and under 25 rpm rotation have the profile shown in FIG. 4. FIG. 4 shows that the dissolution of megestrol acetate suspension is substantially equivalent to that of Par's Megace® ES megestrol acetate suspension.

### Example 11

### Megestrol Acetate Composition

A banana flavored oral suspension containing 125 mg of megestrol acetate per milliliter has the following composition.

| | Quantity | | |
|---|---|---|---|
| Ingredient | per ml | | % w/v |
| Megestrol acetate, micronized | 125 | mg | 12.5 |
| Glycerin | 80 | mg | 8.0 |
| Sodium benzoate | 2 | mg | 0.2 |
| Polyoxyl 35 castor oil | 300 | mg | 30.0 |
| Citric acid | 5.0 | mg | 0.50 |
| Sodium saccharin | 5 | mg | 0.5 |
| Sorbitol solution, 70% | 20 | mg | 2.0 |
| Polysorbate 80 | 50 | mg | 5.0 |
| Banana flavor | 10 | mg | 1.0 |
| Dimethicone | 5.0 | mg | 0.5 |
| Colloidal silicone dioxide | 20.0 | mg | 2.0 |
| Purified water q.s. | 1 | ml | 100 |

What is claimed is:

1. An oral pharmaceutical composition in the form of pharmaceutical elegant and stable flocculated suspension of micronized megestrol acetate, which comprises:
(a) micronized megestrol acetate at a concentration of about 10 to 200 mg per ml;
(b) at least one wetting agent at a concentration of about 0.01 to 55% weight/volume; and
(c) one or two suspending agents at a concentration of about 0.02 to 4% weight/volume, wherein the pH of the suspension can be controlled to 3.0˜4.7 by using an appropriate organic acid.

2. The composition of claim 1 wherein the concentration of micronized megestrol acetate is 10 to 200 mg/ml.

3. The composition of claim 2 wherein the concentration of micronized megestrol acetate is about 125 mg/ml (625 mg/5 ml).

4. The composition of claim 1 wherein the wetting agents have at least one straight carbon chain of up to 40, preferably 4˜40, atoms in the hydrophobic group.

5. The composition of claim 1 wherein the wetting agent is polyoxyl 35 castor oil at a concentration of about 0.05% to 51.5% weight by volume.

6. The composition of claim 5 wherein the concentration of polyoxyl 35 castor oil is 15% or 30% weight by volume.

7. The composition of claim 1 wherein the wetting agent is polyoxyl 40 hydrogenated castor oil at a concentration of about 0.05% to 45% weight by volume.

8. The composition of claim 7 wherein the concentration of polyoxyl 40 hydrogenated castor oil is 10% weight by volume.

9. The composition of claim 1 wherein the wetting agent is polysorbate 80 at a concentration of about 0.01% to 12.6% weight by volume.

10. The composition of claim 9 wherein the concentration of polysorbate 80 is 5% weight by volume.

11. The composition of claim 1 wherein the wetting agent is polyethylene glycol 400 at a concentration of about 0.01% to 5.0% weight by volume.

12. The composition of claim 1 wherein the concentration of polyethylene glycol 400 is 5%, weight by volume.

13. The composition of claim 1 wherein the suspending agents are selected from the group consisting of colloidal silicon dioxide, carboxymethylcellulose sodium, dextrin, gelatin, microcrystalline cellulose, povidone, xanthan gum, tragacanth and a mixture of microcrystalline cellulose and carboxymethyl cellulose sodium.

14. The composition of claim 1 wherein the suspending agent is at a concentration of about 0.2 to 1.5% weight/volume and is a mixture of microcrystalline cellulose and carboxymethyl cellulose, or is at a concentration of 0.1 to 4.0% weight/volume and is colloidal silicone dioxide.

15. The composition of claim 14 wherein the suspending agent is a mixture of microcrystalline cellulose and carboxymethyl cellulose sodium and is at a concentration of 0.8% weight/volume, or is colloidal silicone dioxide at a concentration of is 0.7% or 2.0% weight/volume.

16. The composition of claim 1 which comprises a suspending agent which is colloidal silicone dioxide at a concentration of 0.7% weight/volume, and comprises an anti-foaming agent which is dimethicone at a concentration of 0.5% weight/volume, or which is simethicone at a concentration of 0.5% weight/volume.

17. The composition of claim 1 which comprises pH adjusting agents, sweeteners, flavoring agents and anti-foaming agents.

18. An oral pharmaceutical composition comprising on a percent weight/volume basis megestrol acetate 12.5%, microcrystalline cellulose and carboxymethylcellulose sodium 0.8%, glycerin 8%, sodium benzoate 0.2%, polyoxyl 35 castor oil 15.0%, polyoxyl 40 hydrogenated castor oil 10.0%, sodium saccharine 0.4%, citric acid 0.32%, polysorbate 80 5%, 70% sorbitol solution 20.0%, banana flavor 0.5%, a mixture of dimethicone 0.95% and colloidal silicon dioxide 0.05%, and the remainder purified water, wherein the composition forms a stable flocculated suspension in water.

19. An oral pharmaceutical composition comprising on a percent weight/volume basis megestrol acetate 12.5%, dimethicone 0.7%, colloidal silicone dioxide 0.5%, glycerin 8.0%, sodium benzoate 0.2%, polyoxyl 35 castor oil 15.0%, polyoxyl 40 hydrogenated castor oil 10.0%, sodium saccharine 0.5%, citric acid 0.5%, polysorbate 80 5%, 70% sorbitol solution 2%, banana flavor 1.0%, and the remainder purified water, wherein the composition forms a stable flocculated suspension in water.

20. An oral pharmaceutical composition comprising on a percent weight/volume basis megestrol acetate 12.5%, dimethicone 0.5%, colloidal silicon dioxide 2.0%, glycerin 8.0%, sodium benzoate 0.2%, polyoxyl 35 castor oil 33.0%, sodium saccharine 0.5%, citric acid 0.5%, polyethylene glycol 5%, 70% sorbitol solution 2%, banana flavor 1.0%, and the remainder purified water, wherein the composition forms a stable flocculated suspension in water.

21. An oral pharmaceutical composition comprising on a percent weight/volume basis megestrol acetate 4.0%, dimethicone 0.5%, colloidal silicon dioxide 1.0%, glycerin 8%, sodium benzoate 0.2%, polyoxyl 35 castor oil 12.0%, citric acid 0.5%, sodium saccharine 0.5%, polyethylene glycol 5%, 70% sorbitol solution 2%, banana flavor 1.0%, and the remainder purified water, wherein the composition forms a stable flocculated suspension in water.

22. The composition of according to any one of claims 18-21, wherein the composition has a pH of 3.0 to 4.7.
